(19) United States
(12) Patent Application Publication
(10) Pub. No.: US 2022/0362143 A1
(43) Pub. Date: Nov. 17, 2022

(54) TISSUE DERIVED IMPLANTS REHYDRATABLE WHILE DISPOSED WITHIN A DEVICE

(71) Applicant: Musculoskeletal Transplant Foundation, Edison, NJ (US)

(72) Inventors: Alison Jill Ling, Piscataway, NJ (US); Darrin Nathan Friend, Robbinsville, NJ (US); Scott Tisi Lansdale, Bordentown, NJ (US); Michael Z. Kubik, Randolph, NJ (US)

(21) Appl. No.: 17/728,384

(22) Filed: Apr. 25, 2022

Related U.S. Application Data
(60) Provisional application No. 63/188,245, filed on May 13, 2021.

(51) Int. Cl. A61K 9/00; A61L 31/16; A61L 27/54; A61L 27/58

(52) U.S. Cl. CPC A61K 9/0024; A61L 31/16; A61L 27/54; A61L 27/58; A61F 2250/0068; A61L 2300/414; A61F 2310/00365

(57) ABSTRACT

A tissue derived implant is provided having a configuration which is sized and shaped to be disposed within a reservoir of a handling or storage device, the implant having one or more liquid dispersion features for enabling effective hydration of the implant when the implant is disposed in the reservoir and contacted with a biocompatible liquid. The liquid dispersion features form at least one liquid pathway which facilitates collecting and distributing the biocompatible liquid to contact the substantially the entire implant. An implant assembly is also provided which comprises a handling or storage device comprising an elongated reservoir with the tissue derived implant disposed therein. Additionally, an implant kit is provided which comprises a handling or storage device with an elongated reservoir and the tissue derived implant having an elongated configuration sized and shaped to allow the implant to be disposed in the elongated reservoir at the time of use.

### CROSS-REFERENCE TO RELATED APPLICATIONS

The present application claims the benefit of U.S. Provisional Application No. 63/188,245, filed May 13, 2021, the entire disclosure of which is incorporated by reference herein.

### FIELD

The present invention relates to tissue derived implants which are capable of hydration while disposed in the reservoir of a storage or handling device. More specifically, tissue derived implants, which may be at least partially dehydrated, and have features which enable hydration of the implant while disposed in the reservoir of a device, and prior to displacement from the reservoir to proximate to an implant site of a subject.

### BACKGROUND

Various types of tissue derived implants, which include or are produced from tissue derived matrices, are known and continue to be developed and used to treat many types of conditions in human and non-human patients. Such conditions include, but are not limited to, wounds or otherwise damaged tissue caused by one or more of trauma, disease, or surgical procedures (e.g., therapeutic, reconstructive, or cosmetic), or even by treatments or procedures performed primarily or solely for aesthetic purposes. Treatment generally involves implanting (delivering, placing, positioning, etc.) such a tissue derived implant proximate to (i.e., in or on) an implant site of a subject that has been affected by one or more of the aforesaid conditions.

Tissue derived matrices are typically prepared by processing one or more tissue samples, which have been recovered from one or more donors, using physical or chemical techniques, and often using combinations of both. Further processing, also involving physical or chemical techniques, or both, may be performed to produce a tissue derived implant comprising one or more tissue derived matrices, with or without other additional material and components.

To facilitate storage and transport of tissue derived implants, the processing performed to prepare them (or the tissue derived matrices comprising them) often includes preservation techniques such as freezing, cryopreservation, dehydration, and lyophilization (freeze-drying). One or more of the aforesaid preservation techniques may produce a dehydrated tissue derived implant, which means at least partially dehydrated, and even completely dehydrated. Whether dehydrated or not, tissue derived implants are typically packaged for storage, transport, use, or some combination of these, in a manner and using materials which maintain the condition and properties of the tissue derived implant (e.g., sterility, moisture content, bioactivity, shape, form, etc.) until use. One or more preservation techniques may be applied to the tissue derived matrices before, after, or both before and after, one or more packaging steps or phases, depending on the type of packaging and its components. For example, without limitation, a tissue derived matrix and a preservation agent may be disposed together in a first (e.g., “primary” or “inner”) packaging container which is at least permeable to vaporized water, then subjected to lyophilizing or cryopreserving, and then the first packaging container may be disposed in a second (e.g., “secondary” or “outer”) packaging container which is capable of maintaining the condition and properties of the preserved tissue derived matrix.

It is often desired to hydrate a tissue derived implant, whether dehydrated or not. Generally, hydration of the tissue derived implant is accomplished by contacting or combining the implant with a biocompatible liquid, such as saline, phosphate buffered saline (PBS), blood derived products, antibiotic solution, etc. The implant may be contacted with the biocompatible liquid by any suitable means including, without limitation, one or more of thawing, rinsing, combining, adding, immersing, soaking, and mixing, the implant with one or more biocompatible liquids. Hydrating may be performed or occur prior to or at the time of implantation proximate to an implant site undergoing treatment. Alternatively, or in combination with one or more biocompatible liquids, the tissue derived implant may be hydrated by contact with bodily fluids naturally present at the implant site at the time of or after implantation.

Tissue derived implants are sometimes packaged or disposed in a storage device for short or long term storage, and even for transport (with or without temperature control) or manipulation, prior to use. Sometimes the device is a handling device which facilitates handling, such as one or more of manipulating, mixing, shaping, or delivering (e.g., positioning and depositing) the tissue derived implant proximate to an implant site. Such devices include a reservoir capable of receiving the tissue derived implant and enclosed by a boundary defined by an inner surface of, for example without limitation, a container, tray, jar, or mold with a lid or other cover, a pouch, a cannula, a syringe, etc. Some devices, such as cannulas and syringes have a tube element with an inner surface which defines the boundary enclosing an elongated reservoir, within which the tissue derived implant is disposed during storage, handling or delivery.

It is known to provide a tissue derived implant and device together, either already assembled with the implant disposed in the reservoir of a device. It is also known to provide a tissue derived implant without a device, or with a separate device, in which case a user may place, insert, or otherwise dispose, the tissue derived implant in the reservoir of the device at the time of handling, manipulation, or delivery. The amount or mass of the tissue derived implant which is disposed in the reservoir of a device may be referred to as a dose. In some cases, depending on the size of the implant site, two or more tissue derived implants may be required to effectively treat (fill or partially fill) the implant site, in which case, two or more devices in which a tissue derived implant is disposed (doses) may be employed to treat a single implant site.

When the physical form of the tissue derived implant includes a plurality of elements (e.g., particles, pieces, fragments, elongated elements, fibers, etc.) and the intended use (e.g., size of the implant site to be treated) requires a quantity of such elements of tissue derived implant to be provided and implanted, difficulty may be encountered when loading and disposing the desired quantity of elements of tissue derived implant in a device for storage, transport, or delivery to an implant site. While funnels, tweezers and other implements may be employed to facilitate load the desired quantity of elements of tissue derived implant in the device, this task remains somewhat clumsy and time-consuming, as well as sometimes inefficient and imprecise. This is particularly true when loading the desired quantity of elements of tissue derived implant in the tube element of some devices, such as cannulas and syringes.

Additionally, in cases where a tissue derived implant is hydrated while disposed in the reservoir of a device, by adding a biocompatible liquid to the device, it has been found that unless the implant is packed relatively loosely or with a quantity of biocompatible liquid (e.g., a carrier or preservation agent), effective hydration of the tissue derived implant to produce a flowable implant, does not typically occur within a reasonable time. Thus, it would be advantageous for tissue derived implants to be provided in a form which facilitates substantially complete and effective hydration of the tissue derived implant while disposed in a device such as those mentioned above for storage, handling, delivery, etc., of the implant. It would be further advantageous for the tissue derived implants to have a configuration suitable for disposing or positioning the implant in a storage or handling device. The configuration of the tissue derived implant should also enable, facilitate or even accelerate hydration of the implant while disposed in the device. The invention described and contemplated herein addresses these and other objectives.

### SUMMARY OF THE INVENTION

A tissue derived implant is provided having a configuration which is sized and shaped to be disposed within a reservoir of a handling or storage device and having features which facilitate effective hydration of the implant while disposed in the reservoir. More particularly, the tissue derived implant comprises one or more liquid dispersion features for enabling effective hydration of the tissue derived implant within a reasonable period of hydration time while the implant is disposed in the reservoir and is contacted with a biocompatible liquid. Additionally, the one or more liquid dispersion features form one or more liquid pathways which facilitate collecting and distributing the biocompatible liquid to contact substantially the entire tissue derived implant.

The tissue derived implant may be at least partially dried, lyophilized, or cryopreserved.

In some embodiments, the one or more liquid dispersion features may cooperate with an inner surface of the reservoir to form the one or more liquid pathways while the implant is disposed in the reservoir of the handling or storage device.

In some embodiments, the one or more one or more liquid dispersion features may comprise: a channel, a groove, a notch, a recess, a passage, a lumen, an augmented section, a contracted segment, a recessed segment, a tapered section, a gap, and combinations thereof.

In some embodiments, the tissue derived implant may comprise one or more tissue derived matrices each having a first form comprising one or more of: particulates, fibers, chunks and pieces, and being reshaped into a second form comprising one or more: sheets, blocks, cylinders, plugs, and other three-dimensional shapes

In an exemplary embodiment, the implant may have an elongated configuration comprising a strip having an exterior surface extending a longitudinal distance between opposite first and second ends of the strip, and the one or more liquid dispersion features comprise at least one groove or channel in the exterior surface, each of which extends, independently, at least part of the longitudinal distance. In such an exemplary embodiment, the strip may have an exterior surface extending between opposite first and second ends of the strip, and the one or more liquid dispersion features comprise a plurality of notches on the exterior surface, at least one of which is proximate the first end and at least one other of which is proximate the second end of the strip.

In another exemplary embodiment, the implant may have an elongated configuration comprising a strip or rope and the one or more liquid dispersion features comprise one or more augmented segments, one or more recessed segments, or a combination thereof. In such an embodiment, the implant may have an elongated beaded chain configuration, the one or more augmented sections comprising a plurality of beads and the one or more recessed segments comprising a plurality of constricted bands, and the plurality of beads and the plurality of constructed bands being alternatingly distributed on the implant between a first end of the beaded chain and an opposite end of the beaded chain.

In still another exemplary embodiment, the implant may comprise one or more pieces, each of which includes a first end and the one or more liquid dispersion features comprise an opposite tapered end of smaller diameter than the first end, wherein the pieces are each sized and shaped to allow more than one piece to be disposed in the same reservoir of the storage and handling device and wherein the tapered end of each piece cooperates with an inner surface of the reservoir to form the one or more liquid pathways while the one or more pieces are disposed in the reservoir of the handling or storage device.

An implant assembly is also provided comprising a handling or storage device comprising a reservoir having an inner surface and the tissue derived implant described above is disposed in the reservoir.

An implant assembly is also provided comprising a handling or storage device comprising a tube having an elongated reservoir and the tissue derived implant described above is disposed in the reservoir.

An implant kit comprising a handling or storage device comprising a tube having an elongated reservoir which has an inner surface and the tissue derived implant described above, which comprises an elongated configuration, is sized and shaped to allow the implant to be disposed in the elongated reservoir by a user at the time of use.

### BRIEF DESCRIPTION OF THE DRAWINGS

The present invention will be further explained with reference to the attached drawings, wherein like structures are referred to by like numerals and/or letters throughout the several views. The drawings shown are not necessarily to scale, with emphasis instead generally being placed upon illustrating the principles of the present invention.

FIG. 1A is an elevated perspective view of an exemplary embodiment of a tissue derived implant having a half moon configuration with a liquid dispersion feature comprising a longitudinal groove;

FIG. 1B is a top plan view of the half moon implant of FIG. 1A, clearly showing the groove;

FIG. 1C is a front elevational view of the half moon implant of FIG. 1A, the back elevational view being the same;

FIG. 1D is an elevational end view of the implant of FIG. 1A, also showing the groove;

FIG. 2 is a schematic drawing showing the orientation of a cannula and two half moon implants to be assembled, as well as an enlarged area X2 showing them assembled;

FIG. 3 is a perspective view of a mold device in an assembled state having cavities for producing half moon tissue derived implants;

FIG. 4 is an exploded perspective view of the mold device of FIG. 3;

FIG. 5 is a top plan view of the mold body and end blocks of the mold device of FIG. 4;

FIG. 6A is an elevated perspective view of an exemplary embodiment of a tissue derived implant having a beaded chain configuration with liquid dispersion features comprising alternating beads and recessed segments;

FIG. 6B is front elevational view of the beaded chain implant of FIG. 6A, showing the beads and recessed segments;

FIG. 6C is a top plan view of the beaded chain implant of FIG. 6A, showing the planar face, beads and a recessed segments;

FIG. 6D is an elevational end view of the implant of FIG. 6A;

FIG. 7 is a schematic drawing showing the orientation of a cannula and two beaded chain implants to be assembled, as well as an enlarged area Y2 showing them assembled;

FIG. 8 is a perspective view of a mold device in an assembled state having cavities for producing beaded chain implants;

FIG. 9 is an exploded perspective view of the mold device of FIG. 8;

FIG. 10 is a top plan view of the mold body and end blocks of the mold device of FIG. 8;

FIG. 11A is an elevated perspective view of an exemplary embodiment of a tissue derived implant having a hollow cylinder configuration with a liquid dispersion feature comprising a lumen;

FIG. 11B is an elevational end view of the implant of FIG. 11A, also showing the lumen;

FIG. 12A is an elevated front view of an exemplary embodiment of a tissue derived implant having a bullet configuration with a liquid dispersion feature comprising a tapered end;

FIG. 12B is an elevational end view of the implant of FIG. 12A, looking at the non-tapered end thereof; and

FIG. 13 is a is a schematic drawing showing the orientation of a cannula and several bullet implants to be assembled, as well as an enlarged area Z2 showing them assembled.

### DETAILED DESCRIPTION

The following description explains the present invention and its operation in detail, as well as providing several embodiments. It should be understood that the examples provided herein are exemplary and not intended to limit the scope of the invention described and contemplated hereinbelow. Rather, as will be recognized by persons of ordinary skill in the relevant art, several variations and modifications are possible and within the scope of the invention described and contemplated herein.

It has been recognized that when the tissue derived implant has a mass and physical form which includes a plurality of elements such as, without limitation, particles, pieces, fragments, elongated elements, fibers, etc. (i.e., a first form), loading and disposing the plurality of elements of tissue derived implant in a device for storage, transport or delivery, is often clumsy, time-consuming, inefficient, and imprecise, even using implements such as funnels and tweezers. This is particularly true when loading the desired quantity of elements of tissue derived implant in the tube element of some devices, such as cannulas and syringes. Reshaping or otherwise reforming such a tissue derived implant from a first form (i.e., comprising a plurality of elements) to a second form in which at least a portion of the elements are combined, aggregated, mixed, reshaped, or otherwise assembled, into fewer but larger pieces would facilitate providing a more accurately and consistently measured mass and quantity of tissue derived implant in the device and also reduce the time and effort required to load that implant into the device, either before storage and transport to a user (e.g., medical professional), or after transport but prior to implanting in a patient by a user (e.g., medical professional). For example, without limitation, in some embodiments, at least a portion of the plurality of elements of a tissue derived implant of desired mass and quantity (i.e., a tissue derived implant having a first form or shape), may be combined, aggregated, mixed, reshaped, or otherwise assembled, into up to about 6 pieces (i.e., 1, 2, 3, 4, 5, or 6 pieces), each of which is sized and shaped to enable the entire entire desired mass and quantity of implant material to fit within a device, which can then be loaded and disposed in the device for one or more of storage, transport, and delivery.

It has been found that effective hydration of a tissue derived implant which is disposed within the reservoir of the tube element of typical storage and handling devices (such as cannulas, syringes, etc.), is difficult or not possible, within a reasonable period of hydration time, if the tissue derived implant is packed, loaded, or positioned too tightly or densely in the reservoir. Effective hydration produces a flowable tissue derived implant which is capable of controlled and substantially complete displacement from the device and delivery proximate to an implant site being treated. It is noted that, unless otherwise specifically stated, references hereinafter to “an implant,” “the implant,” “a flowable implant,” and “the flowable implant” all mean “tissue derived implants” in accordance with the description provided herein.

Without wishing to be limited by theory, it is believed that effective hydration is hindered or prevented in the aforesaid circumstances, at least in part, because there are no (or too few, or too small) liquid pathways, i.e., void spaces or regions of decreased density compared to the density of the tissue derived implant, within the elongated reservoir of the tube element that would allow dispersion of biocompatible liquid throughout reservoir and contact with substantially the entire implant. For example, a tissue derived implant may comprise a plurality of smaller pieces, particles, fibers, etc., which have been combined, aggregated, mixed, reshaped, or otherwise assembled, to provide the tissue derived implant having a larger desired shape, as well as having a plurality of pores or void spaces between the constituent pieces, particles, fibers, etc. which may or may not have sufficient size or volume to provide liquid pathways which enable effective hydration of the implant when a biocompatible liquid is contacted with the implant. In such circumstances, depending on how tightly or densely the constituent pieces, particles, fibers, etc. which form the tissue derived implant have been combined, aggregated, mixed, reshaped, or otherwise assembled, the resulting implant may have incidental void spaces (e.g., void spaces, pores, etc. formed as a consequence of using smaller pieces, etc. to form the implant), but they may be too small or have insufficient volume to provide liquid pathways capable of enabling effective hydration of the implant.

Accordingly, again without intending to be bound by theory, it is believed that, if a tissue derived implant is configured to provide or form one or more liquid pathways (i.e., void spaces or regions of decreased density compared to the density of other portions of the derived implant) located or distributed along the length of an elongated reservoir of a device's tube element, when the tissue derived implant is disposed within the reservoir, then biocompatible liquid provided to the reservoir will be able to disperse into or through the liquid pathways and contact the tissue derived implant along the length of the reservoir (e.g., throughout the reservoir). Such broader dispersion of the biocompatible liquid and more thorough contacting of the tissue derived implant, should in turn produce a flowable tissue derived implant capable of substantially complete displacement from the device and controlled delivery (deposition) proximate to an implant site being treated. In some embodiments, where a tissue derived implant includes incidental void spaces as described above, and regardless of whether the incidental void spaces are of sufficient size or volume to enable effective hydration, it is contemplated and within the scope of the presently described invention that such a tissue derived implant would include one or more liquid dispersion features which form one or more liquid pathways which exist in addition to the incidental void spaces. In some embodiments, the liquid dispersion features may augment or supplement any liquid pathways which may be formed by incidental void spaces in or on a tissue derived implant. Furthermore, liquid dispersion features capable of allowing and facilitating dispersion of a hydration liquid throughout a tissue derived implant may comprise one or more regions or portions of different or variable density in or on the tissue derived implant.

As practical matter, under typical conditions which exist at the time of procedures involving implanting tissue derived implants for treatment of an implant site, a “reasonable period of hydration time” is no more than about 60 minutes, which includes any time from about greater than zero seconds to about 60 minutes, for example, no more than about 45 minutes, or no more than about 30 minutes, or no more than about 20 minutes, or no more than about 15 minutes, or no more than about 10 minutes, or no more than about 5 minutes, or no more than 1 minute, or no more than 45 second, or no more than about 30 second, or no more than about 15 seconds, or any other value from greater than zero seconds to about 60 minutes.

As used herein, “hydration” means contact with and absorption of a biocompatible liquid resulting in one or more of decreased viscosity, decreased resistance to displacement, increased flowability, and increased ease of displacement or other movement, of a material such as a tissue derived implant.

Furthermore, “effective hydration” and “effectively hydrated” mean that, when a tissue derived implant is disposed within a tube element of a storage or handling device and a biocompatible liquid is added or provided to the tube element, the biocompatible liquid disperses through the tube element and at least 60% (i.e., any proportion from 60% to 100%), by weight based on the total weight of the tissue derived implant disposed in the tube element is hydrated, extrudable from the tube element, as a cohesive (little to no implant material remaining on hands or gloves of a user) and moldable mass comprising a hydrated tissue derived implant which, when molded retains its shape until intentionally reshaped or re-molded, and when implanted proximate to (i.e., in or on) an implant site of a subject, the hydrated tissue derived implant resists irrigation and migration from the implant site. An implanted hydrated tissue derived implant “resists irrigation and migration from the implant site if less than 25% (i.e., any proportion from 25% to 0%) by weight of the hydrated tissue derived implant (based on the total weight of the hydrated tissue derived implant) migrates or otherwise separates from the implanted hydrated tissue derived implant.

It is possible for a tissue derived implant to be excessively hydrated, which potentially results in issues when displaced from a tube or other device and implanted proximate an implant site, such as falling out of place or otherwise migrating in situ. One exemplary method for using the tissue derived implants described and contemplated herein comprises intentionally providing excess liquid to hydrate the tissue derived implant faster while disposed in a tube or other handling or storage device having a screen, filter or other device at one end or opening thereof that is capable of allowing excess liquid to leave or flow from the handling or storage device while retaining a hydrated tissue derived implant therein. Furthermore, in such an exemplary embodiment, a pushrod, plunger, press plate, or similar device could be inserted at an oppositely oriented end or opening of the tube or other handling or storage device and used to press and squeeze the hydrated tissue derived implant against the filter or screen, to more quickly and efficiently separate excess liquid from the hydrated tissue derived implant, followed by extruding the hydrated tissue derived implant from the handling or storage device to be implanted proximate an implant site, with or without further manipulation, molding, shaping, etc.

As used herein, the term “substantially” means at least 85 percent, or at least 90 percent, or at least 95 percent.

The terms “effective radius” and “effective diameter” are used herein to provide a guide for determining the smallest diameter of elongated reservoir (i.e., inner diameter) of a tube into which a tissue derived implant could be disposed when the tissue derived implant has a variable or irregular radius or diameter, respectively. As used herein, the terms “effective radius” and “effective diameter” mean the greatest radius or diameter, respectively, of the implant being measured. Where a tissue derived implant has a configuration comprising a cylinder with a uniform diameter, the “effective diameter” of such a cylindrical implant would be equal to its diameter. Where a tissue derived implant has a configuration generally comprising a cylinder but with variable diameter, the effective diameter of such an implant would be equal to the greatest diameter of that generally cylindrical implant.

The tissue derived implants described and contemplated herein have a configuration which includes one or more liquid dispersion features which enable effective hydration of the tissue derived implant within a reasonable period of hydration time while disposed in a reservoir of a device. The one or more liquid dispersion features provide one or more liquid pathways facilitate collecting and distributing biocompatible liquid provided to the reservoir, throughout the tube to contact the at least one tissue-derived matrix, thereby enabling and likely accelerating hydration of substantially the entire tissue derived implant. When such a tissue derived implant is disposed in the reservoir of a device, the one or more liquid dispersion features from one or more liquid pathways (i.e., void spaces or regions of decreased density compared to the density of the tissue derived implant), within the reservoir. The liquid pathways enable, and may even accelerate, effective hydration of the tissue derived implant by allowing biocompatible liquid to disperse throughout the reservoir and contact substantially the entire tissue derived implant. The one or more liquid pathways may be in fluid communication, or otherwise interconnected, with one another (e.g., in pairs or collectively), or not.

Generally, the one or more liquid dispersion features of the tissue derived implant may have any configuration (e.g., shape, size, location, etc.) which provides or forms one or more liquid pathways (i.e., void spaces or regions of decreased density compared to the density of the tissue derived implant) in a reservoir or a device while the tissue derived implant is disposed therein. While the one or more liquid pathways are present in the reservoir before biocompatible liquid is provided to the reservoir to hydrate the tissue derived implant therein, in some embodiments, one or more of the liquid pathways may be modified in size or shape, or even become indiscernible to the eye, when the biocompatible liquid is provided or at some time thereafter. Furthermore, as will be described in more detail below, a tissue derived implant may be sized and shaped to be disposed within the reservoir of a tube or other handling or storage device and form one or more liquid dispersion features (which may in turn form one or more liquid pathways) after and while disposed in the tube or other handling or storage device.

The one or more liquid dispersion features may be one or more of any of several types or configurations, for example without limitation, a channel, a groove, a notch, a recess, a passage, a lumen, an augmented section (i.e., “bead” or “knot”), a contracted or recessed segment (i.e., “constricted band”), a tapered section at one or both ends of a tissue derived implant (i.e., a “bullet” or similar shape), and a space or gap between portions, components, or layers of a tissue derived implant, and combinations thereof. It is noted that where a tissue derived implant having more than one liquid dispersion feature of the same type, each liquid dispersion feature may, independently, have the same or different dimensions (e.g., length, depth, width, etc.) as the others. Furthermore, a tissue derived implant may have one or more liquid dispersion features of different types or configurations.

Except for comprising one or more tissue derived matrices and having one or more liquid dispersion features as described above, the composition and configuration (e.g., size, shape, physical features, moisture content, chemical and biological substances, etc.) of tissue derived implants as described and contemplated herein is not particularly limited and will depend, at least in part, upon its intended use and the type devices with which it is intended to be stored, handled, and otherwise used. Similarly, suitable methods for producing the tissue derived implants, having the desired composition and configuration, as well as methods for producing tissue derived matrices included in the tissue derived implant, are not limited and are at the discretion of persons of ordinary skill depending upon the use and devices with which the implants will be stored, handled, and implanted.

For example, without limitation, the tissue derived matrices may be produced from one or more tissue samples selected from: adipose, amnion, artery, bone, cartilage, chorion, colon, dental, dermal, duodenal, endothelial, epithelial, fascial, gastrointestinal, growth plate, intervertebral disc, intestinal mucosa, intestinal serosa, ligament, liver, lung, mammary, meniscal, muscle, nerve, ovarian, parenchymal organ, pericardial, periosteal, peritoneal, placental, skin, spleen, stomach, synovial, tendon, testes, umbilical cord, urological, vascular, vein, and combinations thereof. Consequently, the tissue derived matrices and tissue derived implants comprising them may contain one or more components or derivatives of any one or more of the foregoing tissue types, including without limitation, endogenous cells (viable or not) or their components, growth factors, cytokines, proteins, other bioactive substances, lipids, minerals, extracellular matrix, collagen, fibrin, and the like, and combinations thereof. The processing techniques employed to produce tissue derived matrices may depend upon the type of tissue sample and, as such, may include, without limitation, removal of lipids such as for adipose tissue, demineralizing such as for bone tissue, changes to the molecular structure of collagens or proteins in a tissue sample (e.g., digestion, hydrolysis, cleavage, crosslinking, etc.), and physical or chemical techniques or treatments for increasing or decreasing flexibility, density, compressibility, elasticity, etc., such as are known now or in the future to persons of ordinary skill in the relevant art.

As will be understood and practicable by persons of ordinary skill in the relevant art, the tissue derived matrices, as well as the tissue derived implants comprising one or more of them, may, for example without limitation, be produced or provided in any of the following forms or shapes: particles, strips, chunks, pieces, blocks, sheets, slivers, ribbons, branched and unbranched elongated elements, filaments, fibers, three dimensional geometric shapes such as symmetric and asymmetric spheres, regular and irregular polyhedrons, cones, pyramids, other three dimensional forms having one or more planar or curved surfaces, and irregular three dimensional forms. Additionally, of the foregoing forms and shapes may be combined, aggregated, mixed, reshaped, or otherwise assembled, with one another by any technique or process. In some embodiments, for example without limitation, one or more tissue derived matrices in the form (i.e., having the shape) of particulates, fibers, chunks or pieces, etc. (i.e., a first form or shape) may be molded (reshaped) into another desired shape, for example without limitation, sheets, blocks, cylinders, plugs or other three-dimensional shape (i.e., a second form or shape), such as by manual manipulation, manipulation using a device, or using a mold or other container having a cavity of the desired second form or shape, with or without agitation, drying, freezing, crosslinking, etc. It is noted that, in some embodiments, the second form or shape may comprise a fewer number of larger pieces than in the first form. In some embodiments, the second form or shape may simply be a different shape selected to conform to a known configuration of the reservoir of a storage or handling device.

In some embodiments, the tissue derived implant comprises one or more tissue derived matrices such as, without limitation, freeze-dried viable bone tissue matrix, freeze-dried viable amnion and/or chorion tissue matrix, freeze dried viable cartilage tissue matrix, freeze-dried non-viable bone tissue matrix which comprise cancellous bone, cortical bone, or both, and may be demineralized or not, freeze-dried dermal tissue matrix (viable or not), freeze-dried muscle tissue matrix (viable or not), freeze-dried adipose tissue matrix (viable or not), freeze-dried fascial tissue matrix (viable or not). As described above, any of the foregoing exemplary tissue derived matrices may be in the form of particles, powder, fibers, chips, granules, rods, struts, cylindrical monoliths, etc., and combinations thereof.

As will be recognized by persons of ordinary skill in the relevant art, the tissue derived implant may, optionally, further comprise one or more additional components such as, without limitation: biocompatible fluids, preservation agents (e.g., dimethyl sulfoxide (DMSO), glycerol, glycols, polyols, trehalose, polyphenols such as catechins (e.g., (EGCG)), etc.), carriers (e.g., hyaluronic acid and its derivatives, PBS, etc.), preservatives; antibiotics, and other biocompatible substances; exogenous cells (viable or not), viruses, growth factors, proteins, and other biologically active substances; antioxidants; pharmaceutically active compounds; nutritional substances or media; rheology modifiers; crosslinking agents; pH modifiers (buffers); polymers (natural, synthetic, or both); biologically inert excipients (e.g., calcium carbonate, starch, cellulose, glycol, glycerin, mineral stearates, etc.).

The tissue derived implants described and contemplated herein may have a configuration (i.e., size and shape) which enables the tissue derived implant to be disposed and fit entirely within the reservoir of a device and includes one or more liquid dispersion features as described above. In some embodiments, the tissue derived implant may be a single monolithic piece having the desired configuration and liquid dispersion features, or a combination of multiple smaller shapes (i.e., individual, loose pieces) which have been formed or shaped into a larger configuration having one or more liquid dispersion features.

Methods for producing tissue derived implants having the configurations described herein are not particularly limited. Suitable methods include any shaping or molding techniques and processes known now or in the future to persons of ordinary skill in the relevant art such as, without limitation, casting, molding, machining, printing (e.g., 3-D printing), compressing, cutting, milling, rolling, and combinations thereof. Molding may involve one or more techniques such as, producing or obtaining at least one tissue derived matrix in the form of pieces, particles, fibers powder, etc., optionally collecting, combining, or both, one or more of these matrices and forms together, with or without a biocompatible liquid as a carrier or adhesive, then manually manipulating the resulting tissue derived matrix into a desired shape, or shaping the resulting tissue derived matrix using a mold or similar device having a cavity of the desired shape to receive the tissue derived matrix, optionally followed by one or more of crosslinking, contacting with a preservative agent, freeze-drying, etc.

Several non-limiting exemplary embodiments of tissue derived implants have been contemplated and developed having elongated configurations suitable for disposing in the elongated reservoir of devices which include a tube element (e.g., cannulas, syringes, etc.). Such exemplary embodiments include: a hollow cylinder or strip with a lumen extending from one end to and opposite end thereof; a strip or cylinder with a semi-annular, half moon, or crescent moon cross-section; a strip or cylinder having an exterior surface extending a longitudinal distance between opposite first and second ends and having at least one groove or channel in the exterior surface, each of which extends, independently, at least part of the longitudinal distance; a strip or cylinder having an exterior surface extending between opposite first and second ends and having a plurality of notches on the exterior surface, at least one of which is proximate the first end and at least another of which is proximate the second end of the strip or cylinder; an elongated strip, cylinder, or rope having one or more augmented segments (i.e., “beads” or “knots”), one or more contracted or recessed segments (i.e., “constricted bands”), or a combination thereof, such as alternating beads and constricted bands (also referred to herein as a “beaded chain” or “flexchain” configuration); and a sheet rolled into a cylinder and having one or more gaps or regions of decreased density, compared to the density of the sheet, intermediate adjacent surfaces of the rolled sheet. Any of the foregoing tissue derived implants having elongated configurations may be formed or shaped and either disposed in the reservoir of a device, followed by drying or freeze-drying, or first dried or freeze-dried and then disposed in the reservoir of a device. In some embodiments, the tissue derived implant is partially dried or freeze-dried. In some embodiments, the tissue derived implant is not dried or freeze-dried. In some embodiments, the tissue derived implant is cryopreserved.

It should be noted that any of the liquid pathways formed by one or more liquid dispersion features may be present and recognizable before the implant is disposed within the reservoir of a device. For example, in an embodiment the tissue derived implant having the configuration of a hollow cylinder with a lumen extending from one end to and opposite end of the implant, the lumen is the liquid dispersion feature which provides a void space within the lumen as the liquid pathway, even when the implant is not disposed within the reservoir of a device such as in the barrel of a syringe. Similarly, in another embodiment wherein the tissue derived implant has the configuration of a cylinder or strip with an exterior surface having one or more groves, channels, or notches thereon, these liquid dispersion features provide void spaces as liquid pathways even when the implant is not disposed within the reservoir of a device, although after being disposed in the reservoir, the boundary which defines reservoir further encloses the void spaces, thereby forming enclosed, and more effective, liquid pathways. Other configurations of the tissue derived implants described above, such as the strip or cylinder with a semi-annular, half moon, or crescent moon cross-section, a longitudinal groove, and the beaded chain configuration require being disposed in the reservoir of a device for the boundary to cooperate with the liquid dispersion features (e.g., a longitudinal groove in the planar face of a semi-annular, half moon implant, an indent along the crescent moon implant, the portions of the implant located between adjacent augmented sections (i.e., “beads”), and constricted bands) to further enclose the void spaces and provide enclosed liquid pathways. Additionally, two half moon tissue derived implants may be assembled or combined with one another with their respective planar faces contacting and mated together and their respective longitudinal grooves combined to form a central lumen through the cylinder.

As will be described in further detail hereinbelow, in some embodiments wherein the tissue derived implant is intended to be disposed within an elongated reservoir, such as in a tube element of a device, the tissue derived implant has an elongated configuration with a first end, a second end opposite the first end, and a length extending therebetween, and at least one of the one or more liquid dispersion features is located proximate to the first end of the implant and at least one of the liquid dispersion features is located proximate the second end of the implant, wherein the one or more liquid dispersion features accelerate dispersion of liquid along the length of the implant and hydration of substantially the entire tissue-derived matrix, thereby producing a flowable implant capable of being controllably displaced from the tube.

In some embodiments, the tissue derived implants may be at least partially dried, disposed in a reservoir of a device, and later completely rehydrated while in the reservoir and before displacement from the tube for implanting in a subject.

As used herein, “at least partially dried” means the tissue derived implant has a moisture content of about 75% or less, by weight, based on the total weight of the implant, such as from about 25 wt % to about 65 wt %. In some embodiments, an at least partially dried or “dehydrated” tissue derived implant has a moisture content of from greater than zero to about 75 wt %, such as up to about 70 wt %, or up to about 65 wt %, or up to about 60 wt %, or up to about 55 wt %, or up to about 50 wt %, or up to about 45 wt %, or up to about 40 wt %, or up to about 35 wt %, or up to about 30 wt %, or up to about 25 wt %, or up to about 20 wt %, or up to about 18 wt %, or up to about 15 wt %, or up to about 12 wt %, or up to about 10 wt %, or up to about 9 wt %, or up to about 8 wt %, or up to about 7 wt %, or up to about 6 wt %, or up to about 5 wt %, or up to about 4 wt %, or up to about 3 wt %, or up to about 2 wt %, or up to about 1 wt %.

In some embodiments, the tissue derived implant comprises a first liquid dispersion feature which is located proximate both the first and second ends of the tissue derived implant. Such a first liquid dispersion feature may, for example without limitation, comprise a channel or groove which extends from one end to an opposite end of the implant. In other embodiments, the tissue derived implant comprises one or more liquid dispersion features which are elongated and oriented and positioned along the length of the implant, but extend only a portion of the implant's total length, such as half, one third, one quarter, or less, of the total length of the implant.

In some embodiments, the tissue derived implant comprises a plurality of liquid dispersion features, a first one of which is located proximate a first end of the implant and a second one of which is located proximate a second end of the implant which is opposite the first end. In such embodiments, the plurality of liquid dispersion features may comprise three or more liquid dispersion features wherein at least a third liquid dispersion feature is located at a position intermediate the first and second of the plurality of liquid dispersion features. Such a plurality of liquid dispersion features may, for example without limitation, comprise channels or grooves which do not extend from the first end to the second end, notches, beads, constricted bands, and combinations thereof.

In some embodiments, the tissue derived implant comprises a plurality of liquid dispersion features, a first one of which is located proximate a first end of the implant and a second one of which is located proximate a second end of the implant which is opposite the first end, and wherein the first and second liquid dispersion features are separate and different from one another.

Generally, devices having a reservoir for containing an implant therein are suitable and contemplated for use with the presently described tissue derived implants having one or more liquid dispersion features. For example without limitation, suitable devices include storage devices which are sometimes also or alternatively referred to as “packaging.” Storage devices and packaging are generally configured and designed to contain and store the tissue derived implant for some period of storage time, transport time, or combinations thereof, during which the condition and properties (e.g., sterility, moisture content, bioactivity, shape, form, etc.) of a tissue derived implant are maintained. For example, cannulas are typically storage devices configured and designed to contain, store and transport a tissue derived implant until use. More particularly, a cannula is a tube element with a reservoir therein and which may or may not include additional components (e.g., caps at one or both ends to hold an implant therein).

Additional suitable devices include, for example without limitation, handling devices which are configured and designed to enable any one or more activities involving the tissue derived implant held therein such as, without limitation, containing, handling, transferring, manipulating, positioning, mixing, combining, shaping, reshaping, and delivering. For example, syringes are handling devices configured and designed to enable controlled delivery (i.e., positioning and depositing) of a tissue derived implant proximate an implant site to be treated. In some embodiments, a syringe has a tube element (sometimes referred to as a “barrel”) with the reservoir therein, and a plunger (sometimes referred to as a “piston”) which sealingly fits and reciprocatingly slides within the reservoir for displacing and expelling the tissue derived implant therefrom.

As will be recognized by persons of ordinary skill in the relevant art, storage and handling devices, especially those which include a tube element, may also include or be combined with additional functional elements and devices which enhance the function, facilitate the operation, or both, of storage and handling devices (e.g., to enable or facilitate the addition of a biocompatible liquid to a tissue derived implant disposed therein). Such additional functional elements and devices include, without limitation, luer lock caps, screens, needles, plungers, funnels, flexible or rigid conduits, valves, threaded screw caps, etc.

As will also be recognized by persons of ordinary skill in the relevant art, any particular device having a reservoir may be useful for both storage and handling of a tissue derived implant disposed in the reservoir. For example, without limitation, a cannula may be used for storing, rehydrating, and delivering a tissue derived implant. More particularly, a cannula having a reservoir with a tissue derived implant disposed therein may be transported to a facility where it is stored for a period of storage time. After transport and storage, a user (e.g., a medical professional) may add a biocompatible fluid to the reservoir to hydrate the tissue derived implant and produce a hydrated flowable tissue derived implant. The user may then deliver the flowable tissue derived implant proximate to an implant site undergoing treatment by inserting a dowel, plunger or similar article (which may or may not be provided with the cannula) to displace the flowable tissue derived implant from the reservoir to a position proximate to the implant site. Similarly, a syringe having a tissue derived implant disposed in the reservoir of its barrel may first be stored for a storage period of time before being transported to a facility at which a treatment procedure is performed using the syringe to controllably implant the tissue derived implant proximate an implant site. In such circumstances, both the cannula and syringe were storage and handling devices.

As is determinable be persons of ordinary skill in the relevant art, depending on the intended function of the device and the composition of the particular tissue derived implant to be disposed therein, one or more components of suitable storage and handling devices may be produced from, or include, one or more materials including, without limitation, plastics, polymers (polyethylene terephthalate glycol, (PETG), polypropylene (PP), polyethylene (PE), high density and low density polyethylene (HDPE, LDPE, respectively), polytetrafluoroethylene (PTFE), polyvinylchloride (PVC), cyclic olefin polymers and copolymers (COP, COC, respectively), ACLAR® (and other thermoformable moisture barrier films), nylon, polyester, copolyester, POREX® (and other porous or mesh sheets of PP, PE, ultra-high molecular weight PE (UHMWPE), PTFE, and combinations thereof), isoprene, acrylics, polycarbonate, etc.), glass, metals and alloys thereof (e.g., aluminum, stainless steel, titanium, etc.), TYVEK®, paperboard, and combinations thereof.

As also determinable be persons of ordinary skill in the relevant art, depending on the material of construction and the configuration and intended operation, the device and its components may be produced or formed by various techniques and processes including but not limited to, thermoforming, injection molding, blow molding, die cutting, extruding, film extruding, co-extruding, casting, machining, layering, laminating, film converting, sintering, 3-D printing, knitting, weaving, cutting, carving, folding, and combinations thereof.

In some cases, a tissue derived implant and device are provided together, either already assembled with the implant disposed in the device, or not. In other cases, a tissue derived implant may be provided separately from such a device and a user may place, insert, or otherwise dispose, the tissue derived implant in the device prior to implantation using the device. In some cases, depending on the size of the implant site and the amount or mass of the tissue derived implants disposed in the storage and handling devices (i.e., doses) which is limited by the size of the reservoirs of the respective devices, two or more tissue derived implants may be required to effectively treat (fill or partially fill) the implant site, in which case, two or more devices in which a tissue derived implant is disposed (doses) may be employed to treat a single implant site.

In some embodiments, the tissue derived implant comprises at least one tissue-derived matrix and is disposed in an elongated reservoir of a device having a tube element, the implant having a first end, a second end opposite the first end, and a length extending therebetween, the implant including one or more liquid dispersion features, wherein at least one of the liquid dispersion features is located proximate to the first end of the implant and at least one of the liquid dispersion features if located proximate the second end of the implant, wherein the one or more liquid dispersion features accelerate dispersion of liquid along the length of the implant and hydration of substantially the entire tissue-derived matrix, thereby producing a flowable implant capable of being controllably displaced from the tube element.

With reference now to the figures, although the tissue derived implants will now be described using several exemplary embodiments intended for assembly and use with devices having a tube element with an elongated reservoir for receiving the implant therein, as will be understood by persons of ordinary skill in the relevant art, the present invention is not limited to such embodiments. Rather, there are various other possible embodiments of the tissue derived implants having liquid dispersion features which are contemplated and within the scope of the present detailed description.

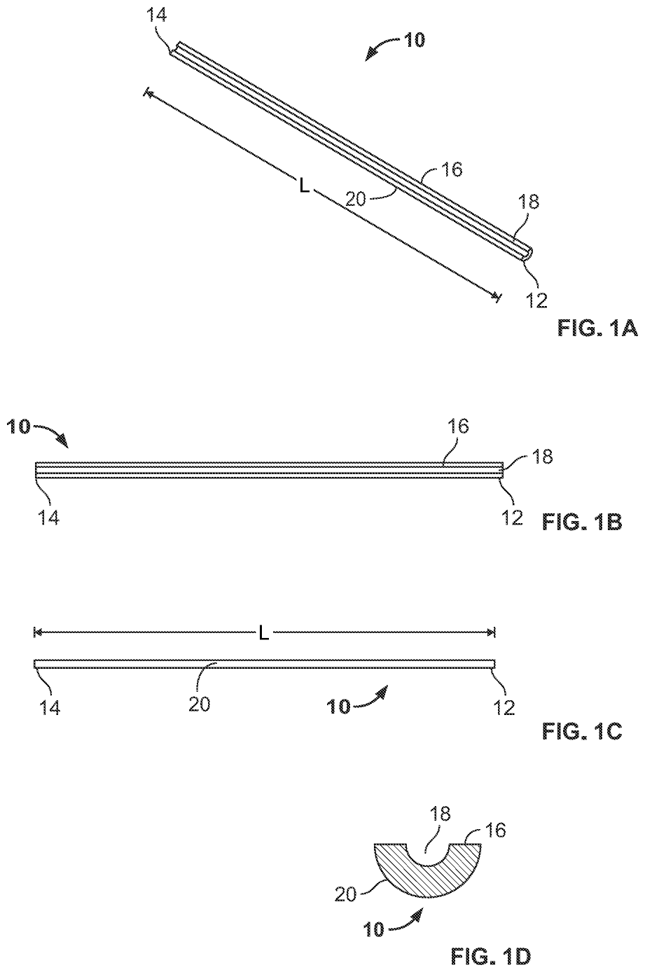

With reference to FIGS. 1A-1D, in some embodiments, the tissue derived implant is a half moon tissue derived implant 10 having an elongated configuration (see FIGS. 1A-1C) with a half moon cross-section, which is most clearly shown in the end view of FIG. 1D. More particularly, such a half moon tissue derived implant 10 includes first end 12, a second end 14 opposite the first end 12, and a length (L) extending therebetween. The half moon tissue derived implant 10 further includes a longitudinal planar face 16 and a liquid dispersion feature comprising a longitudinal grove 18 in the planar face 16 and extending along the length (L), from the first end 12 to the second end 14, of the half moon implant 10. Opposite the planar face 16, the implant also has a curved or semicircular surface 20 having a radius equal to half of the lateral width of the planar face 16. It is noted that in a half moon embodiment of the tissue derived implant 10, the entire longitudinal groove (or grooves, if more than one is provided) 18 defines a void space 18 (see FIGS. 1B and 1D) which is a liquid pathway which enables effective hydration of the implant when a biocompatible liquid is contacted with the implant 10 while disposed in a reservoir, as described below.

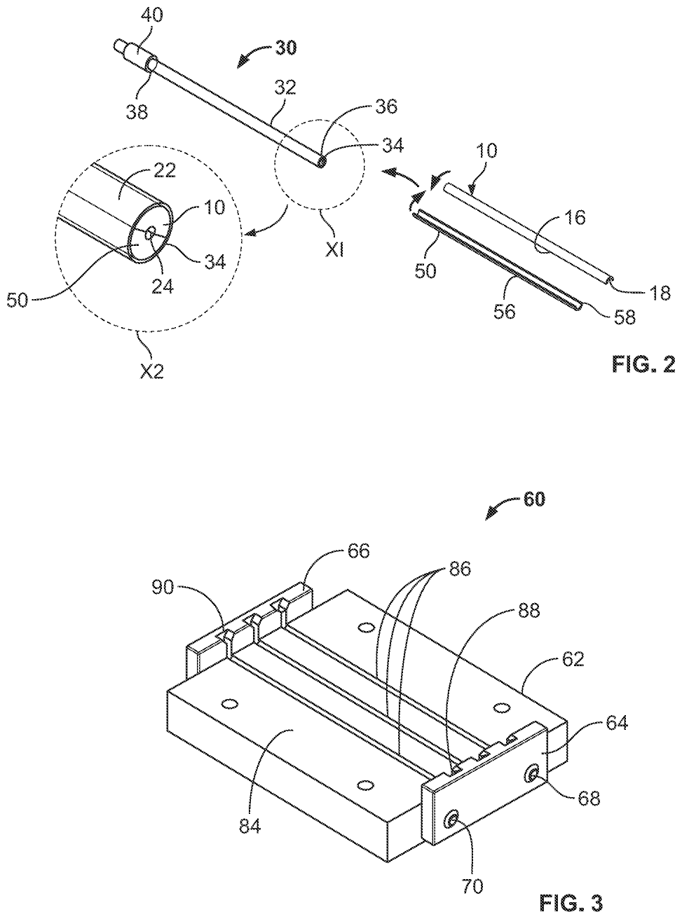

It is noted that, as will be recognized by persons of ordinary skill in the relevant art, in any embodiment of a half moon tissue derived implant 10, the effective radius the implant 10 should be selected and formed depending upon the size of the reservoir into which it is intended to be disposed. In other words, the half moon tissue derived implant 10 should be sized and shaped to have a semicircular surface 20 with an effective radius slightly less than the inner radius of the tube element and its reservoir into which to mated half moon tissue derived implants 10 are intended to be disposed. For example, without limitation, where the inner diameter (i.e., the diameter of the elongate reservoir 34 of the tube element 32) of a cannula 30 is about 6.3 millimeters (mm), the effective radius of each of two half moon tissue derived implants 10, 50 (see FIG. 2) which are to be disposed in such a cannula 30 would most suitably be from about 2.5 to about 2.55 mm, so that when the two half moon tissue derived implants 10, 50 are mated at their planar faces 16, 56 the resulting cylindrical tissue derived implant (see FIG. 2, area X2, described in further detail below) has an effective diameter of up to about 5.1 mm.

Although not shown in particular, in another exemplary alternative embodiment, a tissue derived implant having an elongated configuration may include liquid dispersion features comprising two or more longitudinal grooves which extend only part way along the length of the implant, where one of the longitudinal grooves is proximate the first end of the implant and another of the longitudinal grooves is proximate the second end of the implant. In such embodiments of a half moon tissue derived implant, all of the void spaces provided in the two or more longitudinal groove provide void spaces which are liquid pathways which enable effective hydration of the implant when a biocompatible liquid is contacted with the implant while disposed in a reservoir.

With reference to FIG. 2, the half moon tissue derived implant 10 might, for example, be intended and suitable for use with a device such as a cannula 30 which has a tube element 32 with an elongate reservoir 34 therethrough. To facilitate visualization of how the half moon tissue derived implant 10 may be assembled and disposed in the reservoir 34 of the cannula 30, the cannula shown in FIG. 2 is transparent or translucent. In particular, the tube element 32 includes a first end 36 and a second end 38 opposite the first end 36. The cannula 30 further includes a cap 40 which fits over and sealingly encloses (e.g., by a press fit or threaded connections) the reservoir 34 to retain an implant and other material and substances disposed therein.

With continued reference to FIG. 2, in one exemplary embodiment, two half moon tissue derived implants 10, 50 may be assembled or combined with one another having their respective planar faces 16, 56 contacting and mated together, thereby forming a cylinder shaped implant 22, and having their respective longitudinal grooves 18, 58 combined to form a central lumen 24 through the cylinder shaped implant 22 (see FIG. 2, area X2 which is an enlarged view of area X1 with the two half moon tissue derived implants 10, 50 assembled together and disposed within the reservoir 34 of the cannula 30). The two half moon tissue derived implants 10, 50 may be first be assembled together as described above and then disposed (e.g., inserted manually or with forceps) together in the elongated reservoir 34 of the cannula 30. Alternatively, the two half moon tissue derived implants 10, 50 may each be disposed in the reservoir 34 of the cannula 30, separately, one after the other. With reference to enlarged area X2 of FIG. 2, the lumen 24 formed by the combined longitudinal grooves 18, 58 of the two assembled half moon implants 10, 50, defines a void 24 space which is the liquid pathway which enables effective hydration of the implant when a biocompatible liquid is contacted with the implant while disposed in the reservoir 34.

With reference now to FIGS. 3-5, while it is within the ability of persons of ordinary skill in the relevant art to employ any of various techniques and devices, known now or in the future, to form, assemble, shape, etc., half moon tissue derived implants 10, 50 from one or more tissue derived matrices, an exemplary embodiment of a mold device 60 and process for using it will now be provided. As shown in various views provided in FIGS. 3-5, one exemplary embodiment of a mold device 60, which is useful for producing half moon tissue derived implants, may generally include a mold body 62 and two end blocks 64, 66. The end blocks 64, 66 are assembled and held together, with the mold body 62 therebetween, with threaded bolts or screws 68, 70, 72, 74 inserted through openings 76, 78, 80, 81 which extend through respective end blocks 64, 66, and into threaded openings 82 (only one of which is visible in the figures) provided in the side of the mold body 62.

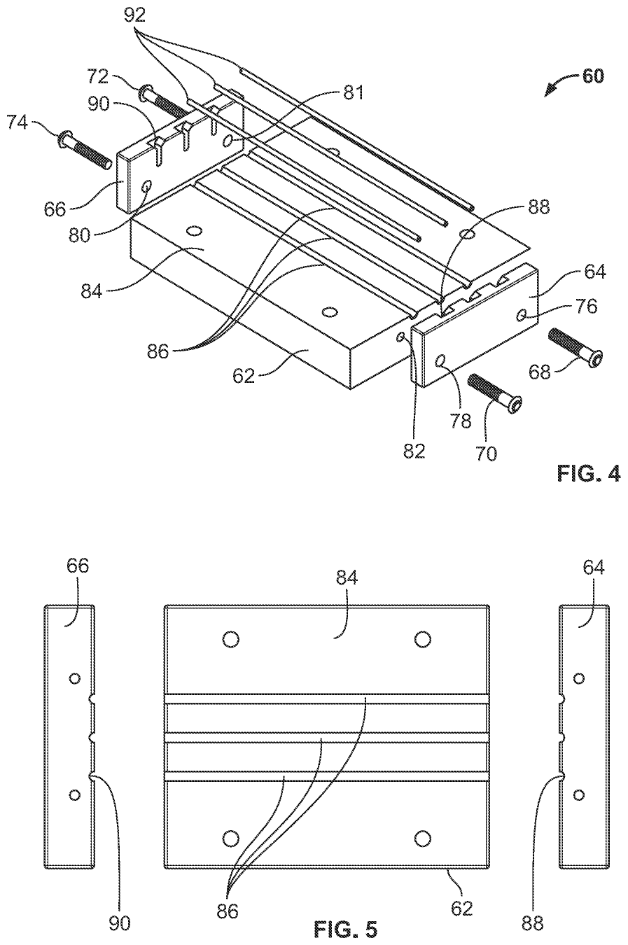

Additionally, the mold body 62 has a substantially planar top surface 84 with one or more grooves or cavities, for example three elongated cavities 86, as shown in FIGS. 3-5. Each of the end blocks 64, 66, has one or more notches, 88, 90, respectively, shown in FIGS. 3-5. Each notch 88, 90 is sized and positioned on its respective end block 64, 66 to align with and extend a respective one of the cavities 86, on the mold body 62. The mold device 60 further includes a rod or pin, such as the three pins 92 shown most clearly in FIG. 4, each of which is sized and shaped to fit into a respective one of the cavities 86, with its ends fitting and held in a pair of aligned notches 88, 90 in the end blocks 64, 66. As described in more detail below, each pin 92, is used to form the liquid dispersion feature (i.e., a longitudinal groove) of each half moon tissue derived implant 10, 50.

It is contemplated that one or more tissue derived matrices (not shown) which are in the form of a flowable or shapeable mass, solution, mixture, putty, etc. comprising a plurality of pieces, particles, fibers, powder, etc., with or without one or more of a biocompatible liquid, a preservation agent, or other additional components, is provided and shaped into one or more half moon tissue derived implants using the mold device 60 as follows. A quantity (e.g., 1.5 grams) of a tissue derived matrix (e.g., bone fibers, mixed or not with bone particles), is placed and spread into each of the cavities 86, such as with a spatula or putty knife, or even manually with fingers. In some embodiments, the one or more tissue derived matrices may further comprise, or be combined with, one or more biocompatible liquids, to increase flowability, prior to being placed into the cavities 86 of the mold device 60. Excess tissue derived matrix which does not fit within a cavity 86 is wiped and cleared from the top surface 84. Each pin 92 is slid or inserted into a pair of aligned notches 88, 90 in the end blocks 64, 66, and then slid and pressed into each cavity 86 and the tissue derived matrix therein, respectively. The entire assembled mold device 60 and tissue derived matrix filling each of its cavities 86 may then be further prepared for subjecting to drying, lyophilizing (freeze-drying), or other process for maintaining the shape of the tissue derived matrix. For example, in an embodiment, a preservative such as 1 milliliter of solution comprising one or more lyophilizing agents (not shown) may be added to each cavity 86, followed by placing the assembled mold device 60 into a Tyvek® film pouch (not shown) and sealed for placement into a lyophilizing apparatus (not shown). The Tyvek® film pouch having the mold device 60 sealed therein is placed into the lyophilizing apparatus and subjected to lyophilizing. Thereafter, each half moon tissue derived implant 10, 50 is removed from its respective cavity 86, and the pin 88 separated therefrom.

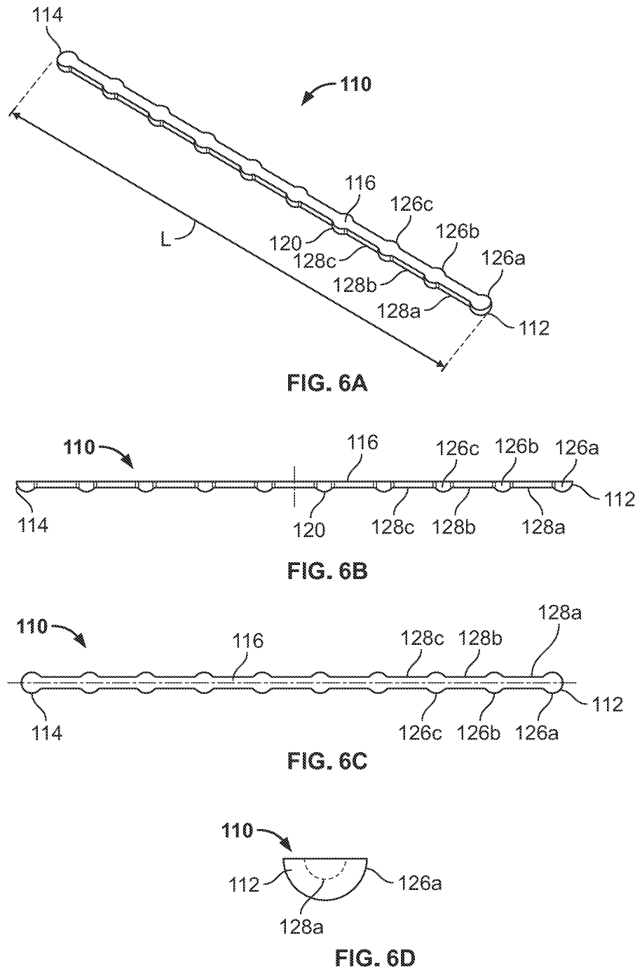

With now reference to FIGS. 6A-6D, in some embodiments, the tissue derived implant is a beaded chain tissue derived implant 110 which also has an elongated configuration (see FIGS. 6A-6C) and has a half circle cross-section (see FIG. 6D), which is most clearly shown in the end view of FIG. 6D. More particularly, such a beaded chain tissue derived implant 110 includes first end 112, a second end 114 opposite the first end 112, and a length (L) extending therebetween. The beaded chain tissue derived implant 110 further includes a longitudinal planar face 116 and one or more liquid dispersion features comprising a plurality of augmented segments, such as the beads 126a, 126b, 126c, and a plurality of contracted sections, such as the recessed segments 128a, 128b, 128c, shown in FIGS. 6A-6C. Additionally, as also shown in FIGS. 6A-6C, the beads 126a, 126b, 126c and recessed segments 128a, 128b, 128c are positioned in alternating arrangement with one another on a curved or semicircular surface 120 opposite the planar face 116 (see the front elevational view of FIG. 6B), and along the length (L), from the first end 112 to the second end 114, of the beaded chain implant 110. The semicircular surface 120 has an effective radius equal to half of the lateral width of a largest one of the beads 126a, 126b, 126c. Although not shown, in another exemplary alternative embodiment, a beaded chain tissue derived implant may have more or fewer beads 126a, 126b, 126c and recessed segments 128a, 128b, 128c as the embodiment described above and show in FIGS. 6A-6D.

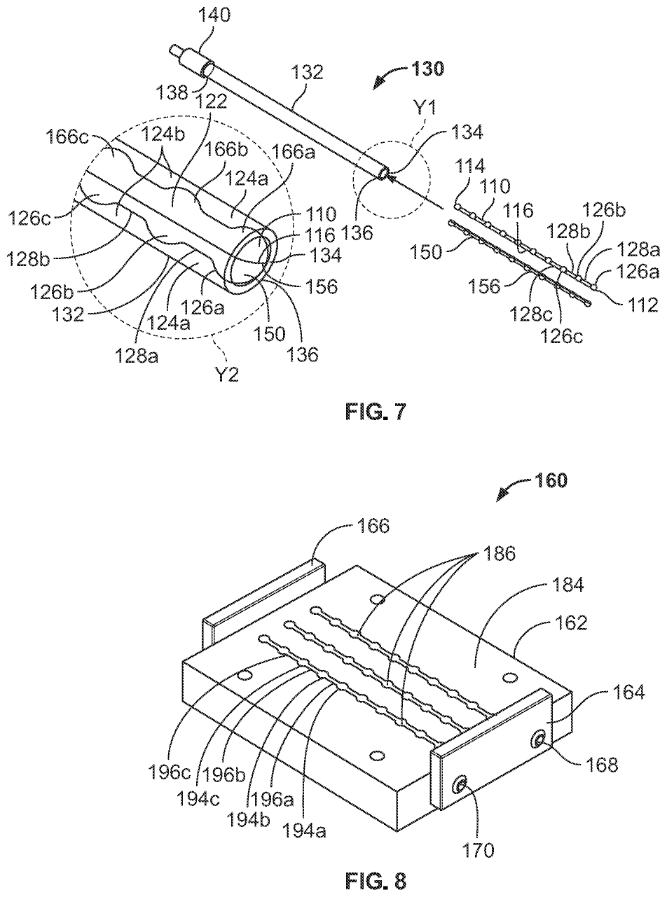

It is noted that, as will be recognized by persons of ordinary skill in the relevant art, in any embodiment of a beaded chain tissue derived implant, the size and shape of the beads 126a, 126b, 126c particularly the effective radius, should be selected and formed depending upon the size of the reservoir into which it is intended to be disposed. In other words, for a beaded chain tissue derived implant 110 which is intended for use with a cannula 130 having an inner diameter o (i.e., the diameter of the elongate reservoir 134 of the tube element 132 of the cannula 130) of about 6.3 mm, which is coverts to an effective radius of about 3.15 mm, each of the beads 126a, 126b, 126c should have an effective radius of no more than about 2.55 mm, such as from about 2.45 to about 2.55 mm. Accordingly, when two such beaded chain tissue derived implants 110, 150 are mated at their planar faces 116, 156 the resulting elongated beaded chain tissue derived implant (see FIG. 7, area Y2, described in further detail below) has an effective diameter of up to about 5.1 mm and will easily fit within the cannula 130 having an inner diameter of about 6.3 mm.

With reference to FIG. 7, the beaded chain derived implant 110 might, for example, be intended and suitable for use with a device such as a cannula 130 which has a tube element 132 with an elongate reservoir 134 as shown previously in FIG. 2. With continued reference to FIG. 7, in one exemplary embodiment, two beaded chain tissue derived implants 110, 150 may be assembled or combined with one another having their respective planar faces 116, 156 contacting and mated together, similar to the two assembled half moon implants 110, 150 discussed above in connection with FIG. 2, thereby forming an elongated beaded chain configuration 122 (see enlarged area Y2, FIG. 7), and having their respective beads 126a, 126b, 126c, 166a, 166b, 166c and respective recessed segments therebetween 128a, 128b, 128c, 168a, 168b aligned in pairs with one another. Enlarged area Y2 in FIG. 7 shows the two beaded chain tissue derived implants 110, 150 assembled together, in an elongated beaded chain configuration 122, and disposed within the reservoir 134 of the cannula 130 (area Y2 is an enlarged view of area Y1 of the empty cannula). The two beaded chain tissue derived implants 110, 150 may be first be assembled together as described above, to form the elongated beaded chain configuration 122, and then disposed (e.g., inserted manually or with forceps) together into the elongated reservoir 134 of the cannula 130. Alternatively, the two beaded chain tissue derived implants 110, 150 may each be disposed in the reservoir 134 of the cannula 130, separately, one after the other. As can be most clearly seen in enlarged area Y2 of FIG. 7, when the two beaded chain tissue derived implants 110, 150 are aligned, assembled and disposed in the reservoir 134 of the cannula 130, multiple void spaces 124a, 124b are formed adjacent the paired recessed segments 128a, 168a, 128b, 168b within the reservoir 134. In this embodiment, these void spaces 124a, 124b provide liquid pathways which enable effective hydration of the beaded chain implants 110, 150 (122) when a biocompatible liquid is provided to the reservoir 134 and contacted with the implants 110, 150 (122).

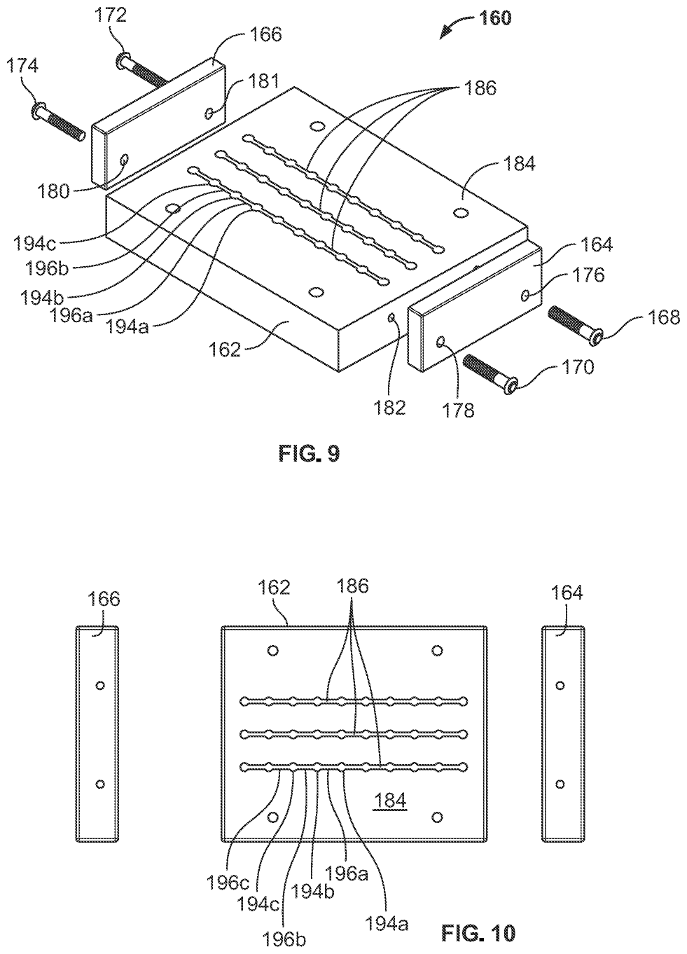

Like the production of tissue derived implants having a half moon configuration (10, 50), methods for producing, forming, assembling, shaping, etc., beaded chain tissue derived implants 110, 150 from one or more tissue derived matrices are not particularly limited. With reference now to FIGS. 8-10, an exemplary embodiment of a mold device 160 for producing such beaded chain implants 110, 150 and a process for using it will now be shown and described. The mold device 160 useful for producing beaded chain tissue derived implants 110, 150 is similar to that described above in connection with FIGS. 3-5 except that the shape of the cavities is different and the end blocks do not need or include notches. Generally, a mold device 160 for producing beaded chain tissue derived implants 110, 150 includes a mold body 162 and two end blocks 164, 166. The end blocks 164, 166 are assembled and held together, with the mold body 162 therebetween, with threaded bolts or screws 168, 170, 172, 174 inserted through openings 176, 178, 180, 181 which extend through respective end blocks 164, 166, and into threaded openings 182 (only one of which is visible in the figures) provided in the side of the mold body 162.

Additionally, the mold body 162 has a substantially planar top surface 184 with one or more grooves or cavities, for example three elongated cavities 186, as shown in FIGS. 8-10. Each of the cavities 186 has alternating widened portions 194a, 194b, 194c, and narrowed portions 196a, 196b, 196c, in between pairs of adjacent widened portions 194a, 194b, 194c, for forming the beads 126a, 126b, 126c and recessed segments 128a, 128b, 128c of the beaded chain implant.

It is contemplated that one or more tissue derived matrices (not shown) which are in the form of a flowable or shapeable mass, solution, mixture, putty, etc. comprising a plurality of pieces, particles, fibers, powder, etc., is provided and shaped into one or more beaded chain tissue derived implants using the mold device 160 in similar manner as described above for the mold device 160 used to produce tissue derived implants having a half moon configuration, (but without pins, of course).

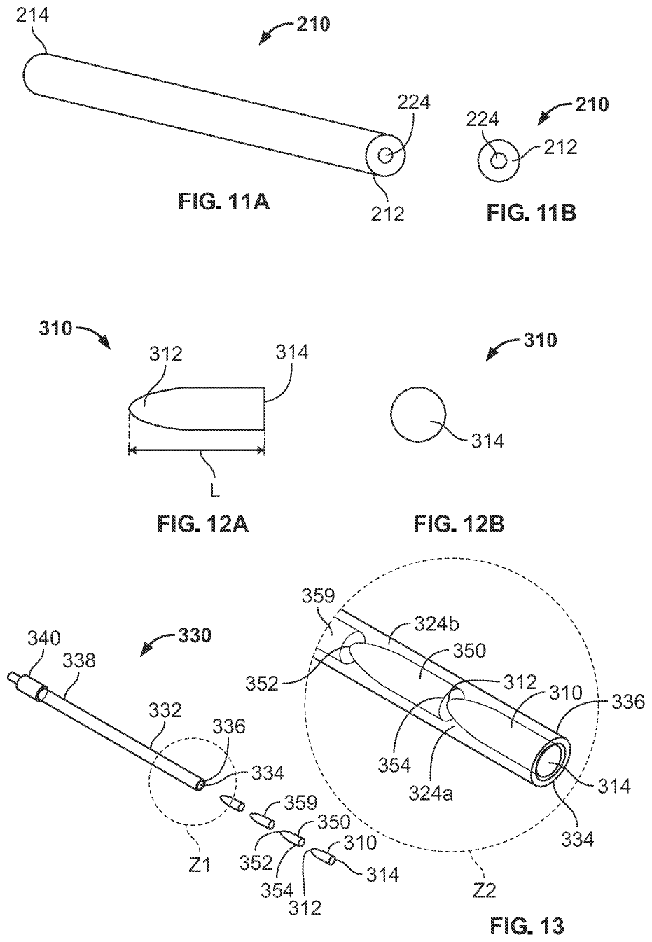

As previously described, another exemplary embodiment of the tissue derived implants having one or more liquid dispersion features would be a hollow cylinder, an example of which is shown in FIGS. 11A and 11B. More particularly, this cylinder implant 210 has an elongated configuration with a lumen 224 extending at least a portion of the length (L) between its first and second ends 212, 214. In the embodiment shown in FIGS. 11A and 11B, the lumen 224 is centrally disposed and extends the entire length (L) of the cylinder tissue derived implant 210. In this cylinder embodiment of the tissue derived implant 210, the lumen 224 defines a void space 224 which is a liquid pathway which enables effective hydration of the cylinder implant 210 when a biocompatible liquid is contacted with the implant 210 in the reservoir of a cannula.

It is noted that, the cylinder tissue derived implant 210 should have an effective diameter selected depending upon the size of the reservoir into which it is intended to be disposed. In other words, a cylinder tissue derived implant 210 should have an effective diameter of from about 5 mm to about 5.1 mm to fit and be disposed in an elongated reservoir of a tube element of a cannula (not shown per se, but see, e.g., FIGS. 2 and 7) or similar device, where the diameter of the elongated reservoir (i.e., the inner diameter of the tube element) is about 6.3 mm.

With reference now to FIGS. 12A, 12B, and 13, another exemplary embodiment of the tissue derived implants having one or more liquid dispersion features would be a bullet, an example of which is shown in FIGS. 12A and 12B. More particularly, this bullet tissue derived implant 310 has a cylinder-like configuration with a relatively short length (L), a first end 312 and a second end 314 opposite the first end 312, where one or both of the first and second ends 312, 314 is tapered or narrowed, wherein each such tapered end is a liquid dispersion feature. In some embodiments in which the tissue derived implant 310 has only one tapered end 312 as shown in FIGS. 12A and 13, the overall shape is a “bullet,” and the non-tapered end 314 defines the effective diameter of the bullet implant (see FIG. 12B). In embodiments having both ends 312, 314 tapered (not shown), then the effective diameter of the bullet tissue derived implant 310 may be defined by a section of the implant 310 intermediate the ends 312, 314 having the greatest diameter.

Enlarged area Z2 in FIG. 13 shows three bullet tissue derived implants 310, 350, 359 disposed within the reservoir 334 of the cannula 330 (area Z2 is an enlarged view of area Z1 of the empty cannula). As can be most clearly seen in enlarged area Z2 of FIG. 13, when the bullet tissue derived implants 310, 350, 359 are disposed in the reservoir 334 of the cannula 330, void spaces 324a, 324b are formed within the reservoir 334, proximate to the tapered end 312, 352 of each bullet implant 310, 350, respectively. In this embodiment, these void spaces 324a, 324b provide liquid pathways which enable effective hydration of the bullet implants 310, 350, 359 when a biocompatible liquid is provided to the reservoir 334 and contacted with the implants 310, 350, 359.

It is noted that the above described half moon, beaded chain and hollow cylinder configurations of the tissue derived implants are generally contemplated as having (but are not required to have) a length (L) which is about equal to or at least 50% of the length of the elongated reservoir into which they are intended to be disposed. The bullet configuration of the tissue derived implants, examples of which are shown in FIGS. 12A, 12B and 13, are generally contemplated as having a length (L) which is not more than about 50% of the of the length of the elongated reservoir into which they are intended to be disposed. Accordingly, two or more bullet tissue derived implants 310, 350 will fit into a reservoir 334 of a storage or delivery device, such as a cannula 330 or syringe (not shown).

Accordingly, as shown in FIG. 13, several bullet implants may be disposed within a reservoir 334 of a cannula 330. Furthermore, bullet tissue derived implants 310, 350, 359 may be each be disposed in the same orientation in the reservoir 334, i.e., tapered end 312, 352 first as shown in FIG. 13, or last. Area Z2 of FIG. 13 is an enlarged view of the empty end of the cannula 330 in area Z1 and shows three bullet tissue derived implants 310, 350, 359 disposed together in the same orientation within the reservoir 334 of the cannula 330. Additionally, it is possible (although not shown) for the bullet implants 310, 350 to be disposed in the reservoir 334 with tapered ends 312, 352 of at least one adjacent pair of bullet implants 310, 350 contacting one another, or with non-tapered ends 314, 354 of at least one adjacent pair of bullet implants 310, 350 contacting one another, or a combination of such orientations.

The bullet tissue derived implant should be sized and shaped, particularly the effective diameter, depending upon the size of the reservoir into which it is intended to be disposed. In other words, a bullet tissue derived implant should have an effective diameter slightly less than the inner diameter of the tube element and its reservoir into which the bullet implant is intended to be disposed. For example, without limitation, where the inner diameter (i.e., the diameter of the elongate reservoir 334 of the tube element 332) of a cannula 330 is about 6.3 mm, the effective diameter of each of the bullet tissue derived implants 310, 350, 359 (see FIG. 13) which are to be disposed in the elongate reservoir 334 of the cannula 330 would most suitably be from about 5 to about 5.1 mm.

Other exemplary embodiments of tissue derived implants having one or more liquid dispersion features may comprise two or more components, having the same or different sizes and shapes as one another. In some embodiments, where at least two of the components have different sizes, different shapes, or both, the liquid dispersion features may form liquid pathways (for example, without limitation, one or more spaces, gaps, reduced density areas, void regions, etc.) when the components are disposed in the reservoir of a cannula, tube element or other handling or storage device.

In another exemplary embodiment, method for producing a tissue derived implant in the shape of a strip or partial cylinder having a crescent moon cross section with a longitudinal indent comprises: disposing an elongated device (e.g., a rod, shaft, bar, etc.) within a cannula or other tube element, wherein the elongated device and tube element have the same length, and the elongated device contacts the inner wall of the tube element along their lengths so as to leave an empty or void region within the tube element having a crescent cross sectional shape, disposing or otherwise placing tissue derived matrix into the tube element in a quantity sufficient to substantially fill the void region, and then lyophilizing the tissue derived matrix while disposed in the tube element with the elongated device. After lyophilizing, the elongated device is withdrawn or removed from the tube element, leaving a tissue derived implant having a crescent moon cross section and a longitudinal indent which extends the length of the implant. The longitudinal indent and inner surface of the tube element form a liquid dispersion feature.

In another exemplary embodiment, tissue derived implants having liquid dispersion features comprising regions of different and varying density of tissue derived matrices are provided. The regions of different and varying density may be positioned in alternating arrangement with one another, or any arrangement such that a hydrating liquid will encounter regions of different density as it disperses along the implant, through the implant, or both. Regions of different density may be formed by placing or disposing tissue derived matrix in different quantities and densities in the reservoir of a tube element or other handling or storage device, or in the reservoir of a mold device such as those described above.

Additionally, the tissue derived matrices used to form the tissue derived implants described and contemplated herein may be disinfected, sterilized, or both, at any time during the processing of the one or more tissue samples recovered from one or more donors to produce the tissue derived matrices and implants comprising them. Furthermore, the tissue derived implants described and contemplated herein may be disinfected, sterilized, or both, at any time or point during the forming, combining, shaping, etc. which is performed to produce the tissue derived implants from one or more tissue derived matrices.

The terms “disinfection” and “disinfecting,” as used herein in all of their grammatical forms, is any process which renders a tissue essentially free of viable pathogenic organisms and viruses by destroying them or otherwise inhibiting their growth or vital activity.

The terms “sterilization” and “sterilizing,” as used herein in all of their grammatical forms, is any process that renders an object (e.g., a tissue, a container for tissue, or an implement for processing tissue) essentially free from pathogenic organisms and/or viruses by destroying them or otherwise inhibiting their growth or vital activity. Such processes may include exposure of the object to one or more, without limitation, of gamma radiation, electron beam radiation, chemical agents (e.g., alcohol, phenol, ethylene oxide gas, acids, bases, or peroxides), heat, or ultraviolet radiation for sufficient duration and dosages. When sterilization is performed on a finished tissue product, such as when disposed in its final packaging, the process may be referred to as “terminal sterilization.”

For example without limitation, disinfection and sterilizing may be performed by contacting (e.g., rinsing, soaking, etc.) one or more tissue samples with a disinfection solution or sterilizing solution, respectively, with or without mechanical agitation. Mechanical agitation increases and enhances the contact between the one or more tissue samples and the disinfecting solution or sterilizing solution, and increases the effectiveness of the disinfecting or sterilizing process, while possible also decreasing the period of time required to achieve an acceptable degree of disinfection or sterilization, respectively. One exemplary disinfection solution would be peracetic acid (0.5%-1%), mild surfactant (0.1% Triton), and buffered saline for debrided bone tissue samples en-block. Another exemplary suitable disinfection solution would be 0.5% to 1.0% peracetic acid in deionized water.

One exemplary suitable sterilizing would include 3% acetic acid in PBS for cancellous bone tissue and 0.6N hydrochloric acid for cortical bone tissue. One or more additional soaks in the disinfecting solution or sterilizing solution may be needed to adequately disinfect or sterilize, respectively, the one or more tissue samples, optionally with the disinfecting solution or sterilizing solution being separated (drained, decanted, etc.) from the tissue and replaced with fresh disinfecting solution or sterilizing solution in between successive soaks. After a final disinfecting or sterilizing soak, the one more tissue samples may be rinsed one or more times in deionized water to remove residual disinfecting solution or sterilizing solution from the tissue sample(s).

It will be understood that the embodiments of the present invention described hereinabove are merely exemplary and that a person skilled in the art may make variations and modifications without departing from the spirit and scope of the invention. All such variations and modifications are intended to be included within the scope of the present invention.

We claim:

1. A tissue derived implant having a configuration which is sized and shaped to be disposed within a reservoir of a handling or storage device, the implant comprising one or more liquid dispersion features for enabling effective hydration of the tissue derived implant within a reasonable period of hydration time when the implant is disposed in the reservoir and is contacted with a biocompatible liquid, wherein the one or more liquid dispersion features form one or more liquid pathways which facilitate collecting and distributing the biocompatible liquid to contact substantially the entire tissue derived implant.

2. The tissue derived implant of claim 1, wherein the one or more liquid pathways are in fluid communication, or otherwise interconnected, with one another.

3. The tissue derived implant of claim 1, wherein the one or more liquid dispersion features cooperate with an inner surface of the reservoir to form the one or more liquid pathways while the implant is disposed in the reservoir of the handling or storage device.

4. The tissue derived implant of claim 1, wherein the one or more one or more liquid dispersion features comprise: a channel, a groove, a notch, a recess, a passage, a lumen, an augmented section, a contracted segment, a recessed segment, a tapered section, a gap, and combinations thereof.

5. The tissue derived implant of claim 4, wherein the implant has an elongated configuration comprising hollow cylinder and the one or more liquid dispersion features comprise a lumen extending from one end of the cylinder to an opposite end thereof.

6. The tissue derived implant of claim 4, wherein the implant has an elongated configuration comprising a strip and the one or more liquid dispersion features comprise a semi-annular cross-section of the strip, a half moon cross-section of the strip, or crescent moon cross-section of the strip, and when disposed within the reservoir of the storage of handling device, the implant occupies a portion of the reservoir leaving an unoccupied portion which forms the one or more liquid pathways.

7. The tissue derived implant of claim 4, wherein the implant has an elongated configuration comprising a strip having an exterior surface extending a longitudinal distance between opposite first and second ends of the strip, and the one or more liquid dispersion features comprise at least one groove or channel in the exterior surface, each of which extends, independently, at least part of the longitudinal distance.

8. The tissue derived implant of claim 4, wherein the implant has an elongated configuration comprising a strip having an exterior surface extending between opposite first and second ends of the strip, and the one or more liquid dispersion features comprise a plurality of notches on the exterior surface, at least one of which is proximate the first end and at least one other of which is proximate the second end of the strip.

9. The tissue derived implant of claim 4, wherein the implant has an elongated configuration comprising a strip or rope and the one or more liquid dispersion features comprise one or more augmented segments, one or more recessed segments, or a combination thereof.

10. The tissue derived implant of claim 9, wherein the implant has an elongated beaded chain configuration, the one or more augmented sections comprise a plurality of beads and the one or more recessed segments comprise a plurality of constricted bands, and the plurality of beads and the plurality of constructed bands are alternatingly distributed on the implant between a first end of the beaded chain and an opposite end of the beaded chain.

11. The tissue derived implant of claim 4, wherein the implant has an elongated configuration comprising a sheet rolled into a cylinder and having one or more gaps or regions of decreased density, compared to the density of the sheet, intermediate adjacent surfaces of the rolled sheet.

12. The tissue derived implant of claim 4, wherein the implant comprises one or more pieces each of which includes a first end and the one or more liquid dispersion features comprise an opposite tapered end of smaller diameter than the first end, wherein the pieces are each sized and shaped to allow more than one piece to be disposed in the same reservoir of the storage and handling device and wherein the tapered end of each piece cooperates with an inner surface of the reservoir to form the one or more liquid pathways while the one or more pieces are disposed in the reservoir of the handling or storage device.

13. The tissue derived implant of claim 1, wherein the implant comprises one or more shapes selected from: particles, strips, chunks, pieces, blocks, sheets, slivers, ribbons, unbranched elongated elements, branched elongated elements, filaments, fibers, three dimensional geometric shapes, symmetric shapes, asymmetric shapes, spheres, regular polyhedrons, irregular polyhedrons, cones, pyramids, three dimensional forms having one or more planar or curved surfaces, and irregular three dimensional forms.

14. The tissue derived implant of claim 1, wherein the implant comprises one or more tissue derived matrices each having a first form comprising one or more of: particulates, fibers, chunks and pieces, and being reshaped into a second form comprising one or more: sheets, blocks, cylinders, plugs, and other three-dimensional shapes

15. The tissue derived implant of claim 1, wherein the implant comprises one or more tissue derived matrices, at least one of which is lyophilized or cryopreserved.

16. The tissue derived implant of claim 1, wherein the implant is at least partially dried.

17. The tissue derived implant of claim 1, further comprising one or more additional components selected from: biocompatible fluids, preservation agents, glycerol, glycols, polyols, trehalose, polyphenols, carriers, preservatives; antibiotics, and other biocompatible substances; exogenous cells, viruses, growth factors, proteins, and other biologically active substances; antioxidants; pharmaceutically active compounds; nutritional substances or media; rheology modifiers; crosslinking agents; pH modifiers; polymers; and biologically inert excipients.

18. An implant assembly comprising a handling or storage device comprising a reservoir having an inner surface and the tissue derived implant of claim 1 disposed in the reservoir.

19. An implant assembly comprising a handling or storage device comprising a tube having an elongated reservoir and the tissue derived implant of claim 1 disposed in the reservoir.

20. An implant kit comprising a handling or storage device comprising a tube having an elongated reservoir which has an inner surface and the tissue derived implant of claim 1, wherein the elongated configuration of the implant is sized and shaped to allow the implant to be disposed in the elongated reservoir by a user at the time of use.
